(19) United States
(12) Patent Application Publication
(10) Pub. No.: US 2006/0094890 A1
(43) Pub. Date: May 4, 2006

(54) Process for producing biodiesel and the product thereof

(76) Inventors: Meeta Sharma, Haryana (IN); Ravindra Kumar, Haryana (IN); Sinha Sabyasachi Ray, Haryana (IN); Rakesh Sarin, Haryana (IN); Ravinder Kumar Malhotra, Haryana (IN); Ram Prakash Verma, Haryana (IN); Niranjan Raje Raghunath, Haryana (IN)

Correspondence Address: NEIFELD IP LAW, PC, 4813-B EISENHOWER AVENUE, ALEXANDRIA, VA, 22304, US

(21) Appl. No.: 11/077,162

(22) Filed: Mar. 11, 2005

(30) Foreign Application Priority Data
Oct. 28, 2004 (IN) 1155/MUM/2004

(51) Int. Cl. C07C 51/43

(52) U.S. Cl. 554/174

(57) ABSTRACT

Disclosed herein is a single pot process for producing biodiesel and the product thereof, using non-edible oil sources containing free fatty acid. The process comprises esterification and transesterification of non-edible vegetable oil sources containing free fatty acids in a single pot employing a water scavenger or a water adsorbent or a mixture thereof.

### FIELD OF THE INVENTION

This invention, in general, relates to an improved process for production of biodiesel and the product thereof. More specifically, but without restriction to the particular embodiments hereinafter described in accordance with the best mode of practice, this invention relates to a single pot process for production of biodiesel using non-edible oil sources containing free fatty acids.

### BACKGROUND OF THE INVENTION

Environmental problems coupled with petroleum reserve depletion stimulated research to develop the renewable transportation fuels. Biodiesel is one of the candidates, which has similar combustion properties as diesel and is being used in a view to reduce the air pollution, to support agriculture and to reduce dependence on the fossil fuel, which are limited resources and localized to some specific regions.

The use of biodiesel in conventional diesel engines results in substantial reduction of un-burnt hydrocarbons, carbon monoxide and particulate matters. Biodiesel is considered as a clean fuel as it has almost no sulphur, no aromatics and has about 10% built-in oxygen, which helps it to burn fully. Its higher cetane number improves the ignition quality even in blends with petroleum diesel.

Fatty Acids Methyl Esters (FAME) have properties very similar to petroleum diesel and so known as biofuel or Biodiesel. These esters can be made from virgin or used vegetable oils or animal fats and can be used as a blend in petroleum diesel.

Processes for producing biodiesel employing different catalysts have been reported in the prior art. These catalysts could be basic, e.g. sodium hydroxide, potassium hydroxide, sodium methoxide, potassium ethoxide or acidic, e.g. sulfuric acid. Biocatalysts like lipases have also been employed for biodiesel synthesis. Solid acid catalysts like alumina and clay have also been used as catalysts in the biodiesel production. Different experimental parameters have been used to develop the process for production of biodiesel.

U.S. Pat. No. 5,525,126 to Basu et al. discloses esterification of a mixture of fats and oils using a calcium acetate-barium acetate catalyst. However, the matter requires elevated temperature, in excess of 200° C., and elevated pressure of approx. 500 psi. These conditions render the esterification process impractical and uneconomical for industrial scale production.

U.S. Pat. No. 5,713,965 to Fogia et al. discloses the use of lipases in the transesterification of triglyceride containing substances and free fatty acids. The conversion rate of only 95% is reported, which cannot yield biodiesel as per the international specifications.

PCT International Application WO 00/05327 to Ginosar et al. discloses use of a critical fluid, high temperature and high pressure to affect a transesterification process.

PCT International Application WO 03/022961 to Bioclean fuels Inc., discloses a process and an apparatus for producing biodiesel by esterifying waste oil with alcohols using static pressure, continuous flow through reaction vessels and specialized reaction tanks with vertical rotating feed tubes.

U.S. patent application Ser. No. 2003/0032826 of Henna discloses a process for the production of fatty acid esters from triglyceride feeds stocks by a process in which the alcohol introduced is being characterized in that having a Reynolds No. of at least about 2100.

U.S. Pat. No. 6,712,867 to Boocock et al. discloses a process for the esterification of triglyceride. The disclosed process comprises of forming a single phase solution of said triglyceride, an alcohol, a base catalyst for the esterification reaction and a co-solvent at a temperature that is less than the boiling point of the solution. The alcohol employed in the process is selected from the group consisting of methanol and ethanol, and mixtures thereof. The ratio of the alcohol to triglyceride is in the range of 15:1 to 35:1. The co-solvent is in an amount sufficient to effect formation of the single phase; permitting esterification to occur in said solution and recovering ester from said solution. The co-solvent is selected from the group consisting of tetrahydrofuran, 1,4-dioxane, diethyl ether, methyltertiarybutylether and diisopropyl ether.

U.S. Pat. No. 6,642,399 to Boocock et al. discloses a single liquid phase process for the esterification of a mixture of fatty acids and triglycerides. The disclosed process comprises of forming a solution of the fatty acids and triglycerides, an alcohol, an acid catalyst, a base catalyst and a co-solvent at a temperature that is less than the boiling point of the solution. The alcohol is selected from the group consisting of methanol, ethanol, and mixtures thereof. The molar ratio of the alcohol to the triglycerides plus one third of the fatty acids is in the range of 15:1 to 35:1. The co-solvent is in an amount to effect formation of a single liquid phase.

U.S. Pat. No. 6,489,496 to Barnhorst et al. discloses that the reaction zone can be any type of vessel commonly used for transesterification reactions, as for example, a reaction vessel having a stirrer or agitator, a vessel having a recirculation loop, or a static mixer within a pipe or a similar container.

U.S. Pat. No. 6,399,800 to Haas et al. discloses a method for producing fatty acid alkyl esters from a feedstock, involving first saponifying the feedstock and then drying the saponified feedstock, and esterifying the dried saponified feedstock with an alcohol in the presence of an inorganic acid catalyst to form fatty acid alkyl esters.

U.S. Pat. No. 6,364,917 to Matsumura, et al. discloses a method and equipment of refining virgin plant oil and/or waste vegetable oil into fuel, preferably diesel engine fuel, by heating the oil, mixing the oil with water and/or ozone and agitating the mixture of oil and water and/or dissipating the ozone.

U.S. Pat. No. 6,768,015 to Luxem et al. discloses a method for making biodiesel from a vegetable oil source, simultaneously reacting the free fatty acids and glycerides of the oil source with methanol, in presence of an acid at temperatures between about 80° C. to about 200° C. and under pressure up to 500 psi.

U.S. Pat. No. 5,302,746 to Koono et al. discloses a process for producing a carboxylic acid ester by reacting a carboxylic acid with an alcohol in the presence of an acid catalyst to produce a reaction solution and neutralizing the reaction solution, using range of aqueous alkali for neutralization.

PCT International Application WO2004081158 discloses a synthetic method for the production of biodiesel from oils and fats. The method discloses that low carbon fatty acid ester ROOR′ in which R and R′ are alkyls with one to four carbons acts as acyl acceptor, transesterification is carried out with bio-lipase, catalyzing the biological oils and fats, the molar ratio of low carbon fatty acid ester and the feeds is 3:1˜20:1. The process also discloses that the by-product glycerine tri- (low carbon) carboxylic ester further reacts with low carbon alcohol R′OH again and said low carbon fatty acid ester can be obtained and recycled for use in the synthesis of the biodiesel.

German Patent No. DE10245758 to Rethmann Klemens et al. discloses a process for production of biodiesel by reacting a branched monohydric alcohol with a fat having a low unsaturated fatty acid content in the presence of sodium hydroxide or potassium hydroxide.

The formation of biodiesel or biofuel by the base catalyzed reaction of vegetable oil with methanol is a two-phase reaction and is known to be slow. Methanolysis is understood to occur only in the methanol phase. Low oil concentration in methanol slows the reaction rate and a slow dissolving rate of the oil in the methanol causes an initiation period. As the concentration increases, the reaction rate also increases, however, the reaction rate subsequently decreases and the reaction tends to stop by completion. This problem gets further aggravated if the vegetable oil contains free fatty acids (FFA). The presence of FFA in the oil leads to formation of soap and emulsion leading to increased processing time and lower yield.

Thus, improvements in the processes for the production of biodiesel are required, in particular for the production of biodiesel from non-edible vegetable oils, which contain substantial amount of free fatty acids. Base catalysed reactions with free fatty acid containing oils lead to formation of excessive emulsions, resulting in lower yield and effluent. Acid catalysed reactions are time consuming.

Therefore, the present invention is directed at a single-pot synthesis process, which produces biodiesel employing a water scavanger or water adsorbents, alone or in a combination thereof, without the process complications of the prior art processes.

### SUMMARY OF THE INVENTION

It is, therefore, an object of the present invention to improve upon the limitations in the prior art. These and other objects are attained in accordance with the present invention wherein there is provided several embodiments of the process for the production of biodiesel comprising esterification and transesterification of non-edible vegetable oils in a single pot employing a water scavanger or water adsorbent.

In one preferred embodiment, there is provided a single pot process for the production of biodiesel, wherein the process comprises esterification of non-edible vegetable oil employing water scavengers or water adsorbents, to trap the byproduct water and facilitate the speedier conversion of free fatty acids present in the non-edible vegetable oil to their methyl esters.

In another preferred embodiment, there is provided a single pot process for the production of biodiesel, wherein the process comprises esterification of non-edible vegetable oil employing a column packed with water adsorbent, to trap the byproduct water and facilitate the speedier conversion of free fatty acids present in the non-edible vegetable oil to their methyl esters.

In another preferred embodiment of the invention, there is provided a single pot process for the production of biodiesel, wherein the process further comprises transesterification of triglyceride to fatty acid methyl ester, wherein the triglyceride is obtained in a mixture of fatty acid methyl ester and triglyceride, after esterification of fatty acids present in non-edible vegetable oil.

In yet another preferred embodiment of the present invention, there is provided an improved one-pot process for the production of biodiesel. The process comprising providing non-edible vegetable oil source having free fatty acid in a reaction vessel, attached with a column or soxhlet apparatus filled with water adsorbent, adding methanol and acidic catalyst in the reactor with stirring, reacting the methanol and the free fatty acid in the presence of an acidic catalyst for an effective conversion of the free fatty acids into fatty acid ester, obtaining the fatty acid methyl ester and triglycerides mixture after the reaction and adding the methanol and a basic catalyst into the same for trans-esterifying triglycerides to fatty acid methyl esters, wherein the traces of acidic catalyst of first esterification step is neutralized with basic catalyst of the second trans-esterification step, yielding water soluble salt, which is washed away during final work-up procedures.

In yet another preferred embodiment of the present invention, there is provided biodiesel produced by a process comprising providing non-edible vegetable oil source having free fatty acid in the reactor attached with the column or soxhlet apparatus filled with water adsorbent, adding methanol and acidic catalyst in a soxhlet apparatus, reacting the methanol and the free fatty acid in the presence of an acidic catalyst for an effective conversion of the free fatty acids into fatty acid ester, obtaining the fatty acid methyl ester and triglycerides mixture after the reaction and adding the methanol and a basic catalyst into the same for trans-esterifying triglycerides to fatty acid methyl esters, wherein the traces of acidic catalyst of first esterification step is neutralized with basic catalyst of the second trans-esterification step, yielding water soluble salt, which is washed away during final work-up procedures.

Furthermore, the present invention also provides recovery of the water adsorbent and reusing the same in the process.

### DETAILED DESCRIPTION OF THE INVENTION

The present invention involves the selection and identification of the plant species capable of giving oil-bearing seeds to produce biodiesel, which can thrive on any type of soil, needs minimum input and post plantation management, and has low moisture demand. Their oil content is quite high about b 25-35%, which justify them to be a choice for energy crops.

Disclosed plant species in the present invention are non-edible vegetable oils selected from the group comprising Jatropha curcas (Ratanjot, Wild Castor, Jangli Erandi), Madhuca indica (Mohuwa) and Pongamia pinnata (Karanj, Honge), Azadiracta indica (Neem), Rice bran etc.

The disclosed embodiment of the present invention deals with a process for the production of biodiesel that has advantages of avoiding lower yield and effluent and provides the time efficient single pot process, which produces biodiesel, without the process complications.

The present invention is directed at a process for the production of fatty acid esters for use as biodiesel, from a feedstock comprising of non-edible vegetable oils from tree borne oil seeds. The tree borne oil contains substantial amount of free fatty acids, which creates emulsification by forming alkali metal salts with the basic catalysts. In acid catalysed processes, the reaction times are quite long, making the process un-economic. According to the present invention, there is provided an improved process where the process is carried out employing both acidic and basic catalysis in a single pot, avoiding the drawbacks of both of them.

Use of a column packed with water adsorbent to trap the byproduct water is an important feature in the present invention. The esterification reaction yields water as a byproduct, which makes the process reversible. In order to direct the reaction in forward direction, the vapors of methanol and water azeotrope are passed through a column, packed with water adsorbent. This facilitates the speedier conversion of free fatty acids to their methyl esters, leaving only fatty acid methyl ester and triglycerides in the reaction mixture.

The water scavanger used in the present invention is selected from 2,2-dimethoxypropane and phosphorous trichloride. The water adsorbent is selected from the group of zeolites, silica gels, acidic clay or molecular sieves. Water scavengers are used insitu in the reaction mixture and water adsorbents are used after filling in the column or soxhlet apparatus. Additionally, water adsorbent can be rejuvenated and recycled.

The intermediate mixture of fatty acid methyl ester and triglyceride obtained after esterification is reacted with methanol in the presence of a basic catalyst. This second step is commenced, only when the intermediate reaction mixture is free from free fatty acids of vegetable oils. This base catalysed reaction trans-esterify triglycerides to fatty acid methyl esters.

In the process of the present invention, free fatty acids present in the starting triglyceride mixture are esterified with a molar excess (relative to the fatty acids) of a lower monoalcohol, i.e., an alkanol having 1 to 5 carbon atoms, in the presence of an acidic esterification catalyst and water scavenger or adsorbent. The preferred alcohol for this pre-esterification step is methanol or ethanol. Comparatively mild reaction conditions are selected for this process, so that transesterification of triglycerides takes place only to a limited extent, if at all it does.

The ratio between free fatty acid (FFA) and methanol is best selected so that, a distinct molar excess of methanol is provided relative to the free fatty acid content to be esterified. Generally, to achieve the results, from about 10% to 50% by volume of methanol is normally used, based on the percentage of FFA in oil. Preferred amounts for this pre-esterification reaction are about 15% to 30% by volume with the most preferred being about 20% by volume. These ratios roughly correspond to molar ratios of methanol (lower monoalcohol) to free fatty acid of about 5:1 to 50:1 depending on the nature and acid number of the triglyceride starting material. Preferably a molar ratio of about 6:1 to 20:1 is employed.

Larger quantities of methanol have a positive effect upon the rate and completeness of the esterification reaction. Even though the solubility of methanol in natural triglycerides is constant for a given reaction temperature, it has been found that, to a certain extent, an increase in the quantity of methanol used produces more rapid and more complete esterification of the free fatty acids.

The reaction temperature can be varied between about the boiling point of the monoalcohol down to about 20° C. below the boiling point. For example, when methanol is used, the reaction temperature should be within the range of about 45° C. to 70° C.

Although the present invention is not intended to be limited to any particular procedure for transesterifying the pre-esterified triglyceride mixture, the anhydrous triglycerides recovered from the process of the present invention preferably are subjected to atmospheric alkali catalyzed transesterification at a reaction temperature in the range of from about 30° C. to 120° C. in a known manner with a lower monoalcohol, e.g., an alkanol having 1 to 5 carbon atoms. The reaction is conducted at atmospheric pressure and it is preferred to carry out the reaction at the reflux temperature of the alcohol employed, e.g., for methanol, at about 65° C., reaction times between about 10 to 60 minutes being typical. Preferred is the same monoalcohol used in the pre-esterification step of the present invention. The most preferred monoalcohol for both steps is methanol and for convenience, the transesterification step will be described briefly with reference thereto.

The transesterification reaction can be carried out batch wise or continuously in any of the many known non-pressurized reaction systems. In general, the methanol is used in a 50% to 150% excess over the stoichiometric quantity required for the transesterification reactions. The transesterification reaction should be carried out with substantially anhydrous methanol. Suitable catalysts for transesterification include alkali metal hydroxides, particularly sodium and potassium hydroxide, and alkali metal alcoholates, particularly sodium methylate. Over and above the quantity required to neutralize any free fatty acids, the catalysts are used in quantities of from about 0.05 to 0.2 percent by weight based on the triglycerides. Preferred are catalyst quantities of from about 0.1 to 0.2 percent by weight, with about 0.15 percent by weight being most preferred.

The following examples are illustrative of the invention and should not be construed as limiting the scope of the invention in any manner. It is understood that the variations of the process described below are possible without departing from the scope and spirit of the invention:

### EXAMPLE-1

The reaction was carried out in the laboratory using a 2000 ml glass reactor, provided with thermostat, mechanical stirring, sampling outlet, and condensation system attached with a 100 ml soxhlet apparatus. The soxhlet apparatus was filled with silica gel (6-20 mesh, 50 g). Jatropha curcas oil (1000 g) having 10% free fatty acid (FFA) was charged to the reactor. Subsequently methanol (175 g) and sulfuric acid (3.0 g) were added slowly to the reactor with stirring. The reaction mixture was refluxed for 1 h through soxhlet apparatus. Reaction mixture was cooled to 50° C. and a solution of sodium hydroxide (9.0 g) in methanol (40 g) was added to the reactor. Temperature of the reactor was raised to 65° C. and refluxing was continued through the soxhlet apparatus for 1 h. The soxhlet apparatus was detached and the excess methanol (80 g) was distilled off. The reaction mixture was transferred to 2000 ml of separating funnel and both the phases were separated. Upper phase was biofuel and lower that of glycerin. The glycerin phase was neutralized with dilute HCl and stored as crude glycerin. Upper phase was washed with hot saline water (2×300 g) to remove the traces of glycerin, catalyst and soap formed during the reaction to yield biodiesel (980 g) meeting the international specifications.

### EXAMPLE-2

The reaction was carried out in the laboratory using a 5000 ml glass reactor, provided with thermostat, mechanical stirring, sampling outlet, and condensation system attached with a 200 ml soxhlet apparatus. The soxhlet apparatus was filled with silica gel (6-20 mesh, 50 g). Karanjia oil (2000 g) having 12% free fatty acid (FFA) was charged to the reactor. Subsequently, methanol (360 g) and sulfuric acid (10.0 g) were added slowly to the reactor with stirring. The reaction mixture was refluxed for 1 h through soxhlet apparatus. Reaction mixture was cooled to 50° C. and a solution of sodium hydroxide (30 g) in methanol (80 g) was added to the reactor. Temperature of the reactor was raised to 65° C. and refluxing was continued through the soxhlet apparatus for one more hour. The soxhlet apparatus was detached and the excess methanol (200 g) was distilled off. The reaction mixture was transferred to 5000 ml of separating funnel and both the phases were separated. Upper phase was biofuel and lower that of glycerin. The glycerin phase was neutralized with dilute HCl and stored as crude glycerin. Upper phase was washed with hot saline water (2×600 g) to remove the traces of glycerin, catalyst and soap formed during the reaction to yield biodiesel (1950 g) meeting the international specifications.

### EXAMPLE-3

The glass reactor was used as given in Example-2. The soxhlet apparatus was filled with silica gel (6-16 mesh, 70 g). Mohua oil (2000 g) having 15% FFA was charged to the reactor. Subsequently methanol (380 g) and sulfuric acid (12.0 g) were added slowly to the reactor with stirring. The reaction mixture was refluxed for 1 h through soxhlet apparatus. Reaction mixture was cooled to 50° C. and a solution of sodium hydroxide (25 g) in methanol (105 g) was added to the reactor. The reaction mixture was heated at reflux through the soxhlet apparatus for 1 h. The soxhlet apparatus was detached and the excess methanol (200 g) was distilled off. The work-up process was followed as given in Example-1 to yield biodiesel (1940 g).

### EXAMPLE-4

The glass reactor was used as given in Example-2. The soxhlet apparatus was filled with silica gel (6-16 mesh, 70 g). Karanjia oil (2000 g) having 15% FFA was charged to the reactor. Subsequently methanol (400 g) and p-toluene sulphonic acid (18.0 g) were added slowly to the reactor with stirring. The reaction mixture was refluxed for 1 h through soxhlet apparatus. Reaction mixture was cooled to 50° C. and a solution of sodium hydroxide (25 g) in methanol (105 g) was added to the reactor. The reaction mixture was heated at reflux through the soxhlet apparatus for 1 h more. The soxhlet apparatus was detached and the excess methanol (200 g) was distilled off. The work-up process was followed as given in Example-1 to yield biodiesel (1930 g).

### EXAMPLE-5

The glass reactor was used as given in Example-2. A silica gel packed glass column was used in place of the soxhlet apparatus. Karanjia oil (2000 g) having 15% FFA was charged to the reactor. Subsequently methanol (400 g) and p-toluene sulphonic acid (18.0 g) were added slowly to the reactor with stirring. The reaction mixture was refluxed for 1 h through silica gel column. Reaction mixture was cooled to 50° C. and a solution of sodium hydroxide (25 g) in methanol (105 g) was added to the reactor. The reaction mixture was heated at reflux through the soxhlet apparatus for 1 h. The glass reactor was isolated and the excess methanol (200 g) was distilled off. The work-up process was followed as given in Example-1 to yield biodiesel (1950 g).

### EXAMPLE-6

The glass reactor and silica column were used as in Example-5. Mohua oil (3000 g) having 17% FFA was charged to the reactor. Subsequently, methanol (550 g) and p-toluene sulphonic acid (30.0 g) were added slowly to the reactor with stirring. The reaction mixture was refluxed for 1 h as given in Example-3. Reaction mixture was cooled to 50° C. and a solution of Potassium hydroxide (40 g) in methanol (155 g) was added to the reactor. The reaction mixture was heated at reflux through the soxhlet apparatus for more 1 h. The soxhlet apparatus was detached and the excess methanol (310 g) was distilled off. The work-up process was followed as given in Example-1 to yield biodiesel (3860 g).

### EXAMPLE-7

The glass reactor and silica column were used as in exmple-5. Neem oil (3000 g) having 18% FFA was charged to the reactor. Subsequently, methanol (600 g) and sulfuric acid (22.0 g) were added slowly to the reactor with stirring. The reaction mixture was refluxed for 1 h as given in Example-1. Reaction mixture was cooled to 45° C. and a solution of Potassium hydroxide (45 g) in methanol (180 g) was added to the reactor. The reaction mixture was heated as given in Example-1 and excess methanol (300 g) was distilled off. The work-up process was followed as given in Example-1 to yield biodiesel (2850 g).

### EXAMPLE-8

The glass reactor was used as given in Example-2. The soxhlet apparatus was filled with silica gel (6-16 mesh, 90 g). Rice Bran oil (2000 g) having 15% FFA was charged to the reactor. Subsequently, methanol (400 g) and sulfuric acid (30 g) were added slowly to the reactor with stirring. The reaction mixture was refluxed for 1 h as given in Example-1. Reaction mixture was cooled to 40° C. and a solution of Potassium hydroxide (20 g) in methanol (120 g) was added to the reactor. The reaction mixture was heated as given in Example-1 and excess methanol (240 g) was distilled off. The work-up process was followed as given in Example-1 to yield biodiesel (1940 g).

### EXAMPLE-9

The glass reactor was used as given in Example-2. The soxhlet apparatus was filled with anhydrous activated acidic clay (100 g). Rice Bran oil (2000 g) having 15% FFA was charged to the reactor. Subsequently, methanol (400 g) and sulfuric acid (30 g) were added slowly to the reactor with stirring. The reaction mixture was refluxed for 1 h as given in Example-1. Reaction mixture was cooled to 40° C. and a solution of Potassium hydroxide (20 g) in methanol (120 g) was added to the reactor. The reaction mixture was heated as given in Example-1 and excess methanol (240 g) was distilled off. The work-up process was followed as given in Example-1 to yield biodiesel (1940 g).

### EXAMPLE-10

The reaction was carried out in the laboratory using a 5000 ml glass reactor, provided with thermostat, mechanical stirring, sampling outlet, and condensation system. Karanjia oil (2000 g) having 12% free fatty acid (FFA) was charged to the reactor. Subsequently, methanol (360 g) and sulfuric acid (10.0 g) were added slowly to the reactor with stirring followed by 2,2-dimethtoxypropane (20 g). The reaction mixture was refluxed for 1 h. Reaction mixture was cooled to 50° C. and a solution of sodium hydroxide (30 g) in methanol (80 g) was added to the reactor. Temperature of the reactor was raised to 65° C. and refluxing was continued for more 1 h. The excess methanol (200 g) was distilled off. The reaction mixture was transferred to 5000 ml of separating funnel and both the phases were separated. Upper phase was biodiesel and lower that of glycerin. The glycerin phase was neutralized with dilute HCl and stored as crude glycerin. Upper phase was washed with hot saline water (2×600 g) to remove the traces of glycerin, catalyst and soap formed during the reaction to yield biodiesel (1950 g) meeting the international specifications.

Certain modifications and improvements of the disclosed invention will occur to those skilled in the art without departing from the scope of invention, which is limited only by the appended claims.

We claim:

1. A single pot process for producing a biodiesel, the process comprising mixing a feedstock with an alcohol and an acidic catalyst in the presence of a water scavenger or water adsorbent for effective esterification of free fatty acid to fatty acid ester, wherein the fatty acid ester is obtained as a mixture of fatty acid ester and triglycerides, adding an alcohol and a basic catalyst into the resultant mixture to trans-esterify the triglycerides to fatty acid ester, wherein the fatty acid ester thus produced is capable of being used as biodiesel.

2. The process according to claim 1, wherein the feedstock includes free fatty acid concentration of more than 1%.

3. The process according to claim 1, wherein the feedstock is non-edible vegetable oil sources.

4. The process according to claim 3, wherein the non-edible vegetable oil sources are plant species selected from the group comprising Jatropha, Pongamia, Madhuca indica, Neem or Rice bran oil.

5. The process according to claim 1, wherein the water scavenger is selected from a group comprising 2,2-dimethoxypropane and phosphorous trichloride.

6. The process according to claim 1, wherein the water adsorbent is selected from the group comprising zeolites, silica gels, acidic clays or molecular sieves.

7. The process according to claim 1, wherein the alcohol is methanol or ethanol.

8. The process according to claim 1, wherein the acidic catalyst is sulphuric acid.

9. The process according to claim 1, wherein the basic catalyst is selected from sodium hydroxide, potassium hydroxide, sodium methoxide or potassium ethoxide.

10. The process according to claim 1, wherein the acidic catalyst used in the esterification step is neutralized with the basic catalyst used in the trans-esterification step.

11. The process according to claim 1, wherein said biodiesel is capable of augmenting the lubricity of petroleum diesel.

12. A process for producing a biodiesel, the process comprising:
a) providing a feedstock of non-edible vegetable oil source having free fatty acid in a reaction vessel attached with a column or soxhlet apparatus filled with a water adsorbent;
b) adding an alcohol and an acidic catalyst in the reaction vessel;
c) reacting the alcohol and the free fatty acid in the presence of an acidic catalyst to obtain a mixture of fatty acid ester and triglycerides;
d) adding an alcohol and a basic catalyst into the same to trans-esterify the triglycerides to fatty acid esters, wherein the fatty acid ester thus produced is capable of being used as a biodiesel.

13. The process according to claim 12, wherein the non-edible vegetable oil sources are plant species selected from the group comprising Jatropha curcas, Pongamia pinnata, Madhuca indica, Neem or Rice bran.

14. The process according to claim 12, wherein the water adsorbent is selected from a group comprising zeolites, silica gels, acidic clays or molecular sieves.

15. The process according to claim 12, wherein the alcohol is methanol or ethanol.

16. The process according to claim 12, wherein the acidic catalyst is sulphuric acid, Hydrochloric acid or p-toluene sulphonic acid.

17. The process according to claim 12, wherein the basic catalyst is selected from sodium hydroxide, potassium hydroxide, sodium methoxide or potassium ethoxide.

18. The process according to claim 12, wherein the acidic catalyst used in the esterification step is neutralized with the basic catalyst used in the trans-esterification step.

19. The process according to the claim 12, wherein the process is a single pot process.

20. A biodiesel produced by a process comprising:
a) providing a feedstock of non-edible vegetable oil source having free fatty acid in a reaction vessel, attached with a column or soxhlet apparatus filled with a water adsorbent;
b) adding an alcohol and an acidic catalyst in the reaction vessel;
c) reacting the alcohol and the free fatty acid in the presence of an acidic catalyst to obtain a mixture of fatty acid ester and triglycerides;
d) adding an alcohol and a basic catalyst into the same to trans-esterify the triglycerides to fatty acid esters, wherein the fatty acid ester thus produced is capable of being used as a biodiesel.
